(19) United States
(12) Patent Application Publication
(10) Pub. No.: US 2013/0245661 A1
(43) Pub. Date: Sep. 19, 2013

(54) Vaginal Stent and Method of Insertion

(71) Applicant: Rita Angel, New York, NY (US)

(72) Inventor: Albert Altchek, New York, NY (US)

(21) Appl. No.: 13/423,373

(22) Filed: Mar. 19, 2012

(51) Int. Cl. A61M 29/02

(52) U.S. Cl. 606/191

(57) ABSTRACT

In one example, a vaginal stent having a top and a bottom and includes from a proximal end of the stent to a distal end of the stent, a knob portion, a neck portion which adjoins a body portion in a shoulder region, and a body with a top surface and a bottom surface. In the foregoing stent, a periphery of a cross section of the body is taken midway along the body along a longitudinal axis of the body, in a direction from the proximal end to the distal end of the stent, defining a flattened shape having a major axis and a minor axis. An associated method of using the stent is also disclosed.

### FIELD OF THE INVENTION

The field relates to vaginal stents and associated methods of using vaginal stents for bodily orifices.

### BACKGROUND OF THE INVENTION

Stents for use in gynecological examination are known in the art but have been plagued by difficulty of use due to their geometry and other factors.

The deceased inventor of this specification, in writing a chapter for a medical treatise, entitled, “Pediatric Adolescent and Young Adult Gynecology,” described such known problematic stents for dilating the vaginal canal. For example, the Counseller stent, now no longer available, was a hard, plastic, torpedo-shaped stent which included a knob for protrusion through the vaginal orifice. This stent had many problems such as its length, excessive weight, and pressure on the vault apex. Yet another stent, the Jonas stent, included a round cross-section and a slight bottle shape with a knob. Its bottle shape helped aided in preventing expulsion and is easier for patients to handle. However, its round cross-section causes the stent to press on the bladder and may cause discomfort as well as increasing the chance of urinary tract infection. Additionally, the round cross-section also caused the stent to press on the section behind it, causing discomfort and a sensation of having stool in the rectum even after defecation. Patients also had to hold the stent and thus, the stent's use was limited to a few minutes. Yet another stent, a British stent made by the Rocket of London company is a flat teardrop-shaped stent made of a clear, hard, plastic, but did not include a handle. Although this stent is easy-to-use and is self-retaining, it was difficult to remove without drilling a hole into the neck of the stent. The inventor, by using that method, subsequently was able to remove the stent.

In the aforementioned treatise, the inventor further mentions various features of a potential stent, but without illustration or sufficient verbal detail so as to leave such teaching insufficient in various respects.

There remains a need for an improved vaginal stent which is more effective, easier to use and one that reduces pain and physiological harm to a patient.

### SUMMARY OF THE INVENTION

In one example, a vaginal stent having a top and a bottom and includes from a proximal end of the stent to a distal end of the stent, a knob portion, a neck portion which adjoins a body portion in a shoulder region, and a body with a top surface and a bottom surface.

In the foregoing stent, a periphery of a cross section of the body is taken midway along the body along a longitudinal axis of the body, in a direction from the proximal end to the distal end of the stent, defining a flattened shape having a major axis and a minor axis.

The foregoing stent fulfills the need for an improved vaginal stent which is more effective, easier to use and one that reduces pain and physiological harm to a patient.

In one example, an associated method of using the vaginal stent includes the steps of first inserting the distal end of the stent into the a vaginal orifice of a human female with said major axis of the stent body being sufficiently angled from an approximately linear orifice of an empty vagina such that the knob avoids pressing a female urethra; further moving the stent backwardly into the vaginal canal until the neck portion becomes positioned at the vaginal orifice and the stent extending for a length into the vaginal canal such that the stent becomes locked in place by having the shoulders of the neck portion being self-retained and supported by levator ani muscles and fascia of the vaginal canal; and further positioning the stent such that the stent is substantially horizontally aligned with the transverse area of the vaginal canal by rotating of the knob.

The foregoing method of insertion facilitates an exemplary manner of using the stent while avoiding discomfort to the user during the insertion process.

### BRIEF DESCRIPTION OF THE DRAWINGS

Other features and advantages of the invention will become apparent from reading the following description in conjunction with the following drawings, which are drawn to scale, and in which like reference numerals refer to like parts:

FIGS. 1 and 2 are top and side views of one example of the vaginal stent of the present invention.

FIG. 3 is a rotated view of the vaginal stent of FIG. 2.

FIG. 4 is a cross-sectional view of the vaginal stent of FIGS. 1 and 2 and surrounding anatomy of a female person when the stent is fully and properly inserted.

FIG. 5 is front view of the stent of FIGS. 1 and 2.

FIG. 6 is a cross-sectional view of the periphery of the stent body of FIG. 1, taken at the arrows marked FIG. 6 in FIG. 1 mid-way along a longitudinal axis of the stent body.

FIG. 7 is a simplified side view of stent 10, showing modifications to curvatures of the neck portion.

FIG. 8 is a cross-sectional of an empty vaginal canal and surrounding anatomy of a female person.

FIGS. 9-11 are schematic, partial cross-sections at the respective depths into a vaginal canal at the arrows marked FIG. 9, FIG. 10, and FIG. 11 in FIG. 8, with each of FIGS. 9-11 showing on the left an empty vaginal canal and on the right a vaginal canal with a non-cross-sectioned stent at a typical orientation during the process of inserting the stent into the vaginal canal.

FIG. 12 is a view of a posterior cut surface of the female pelvis in the area of the vagina and the uterus taken at the arrows marked FIG. 12 in FIG. 8.

### DETAILED DESCRIPTION

The examples and drawings provided in the detailed description are merely examples, and should not be used to limit the scope of the claims in any claim construction or interpretation.

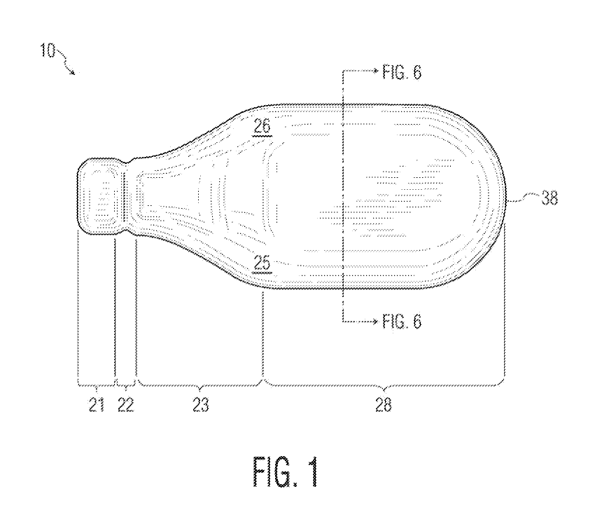

FIGS. 1 and 2 show top and bottom views of a vaginal stent 10, which is intended to be inserted into a vaginal canal of a female person for the purpose of dilating the vaginal to increase its size, for purposes in treatment, among other uses.

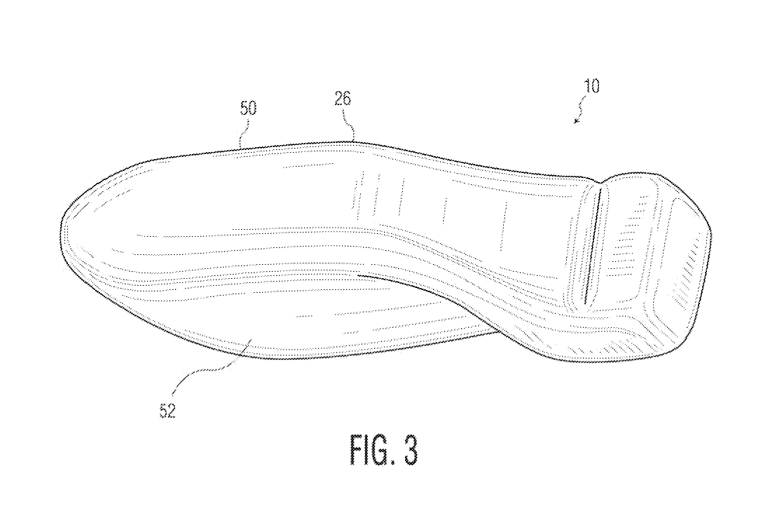

Before detailing features of the stent 10 of FIGS. 1 and 2, FIG. 4 shows the stent 10 inserted into a vaginal canal 13, and the relation of the inserted stent to anatomical features such as a urethra 16 adjacent a top surface of the stent and a rectum 19. Other anatomical features in FIG. 4 and their relation to stent 10 will be described below.

Referring to FIGS. 1 and 2, stent 10 includes from the left-shown end to the right-shown end, a knob portion 21, a neck portion 23 and a body 28. As used herein, the left-shown end of the stent is referred to as the proximal end of the stent, and the right-shown end is referred to as the distal end of the stent.

Preferably, a transition depression portion 22, such as a groove, is provided between the knob portion 21 and the neck portion 23. Neck portion 23 includes a pair of shoulder regions 25 and 26. A stent end portion 38 is shown.

Stent 10 may be made of plastic, acrylic, or SILASTIC-brand material, and combinations thereof, for example. Other materials for stents will be routine to those of ordinary skill in the art based on the present specification.

Stent body 28 is shaped to accommodate a vaginal canal. For instance, in a preferred embodiment, the distal end of body 28 is curved to match an inner surface of the vaginal canal, and neck 23 gradually decreases in width from body 28 to knob 21 to match the circumference of a vaginal canal, which is reduced at a lesser depth.

### Transition Depression Portion

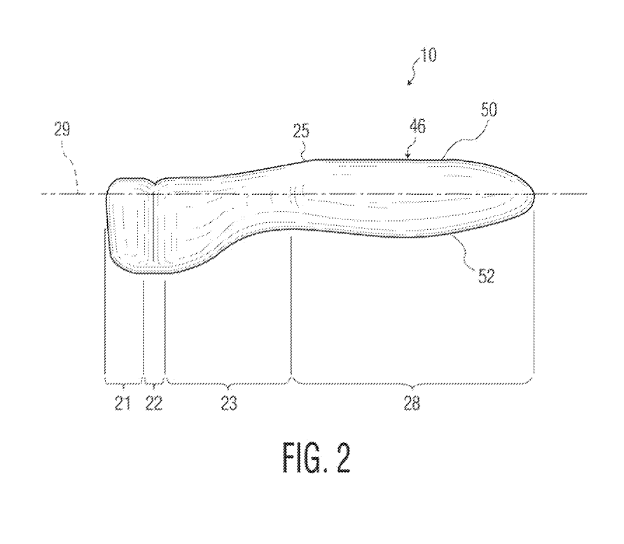

The preferred transition depression portion 22 assists gripping of the stent 10 by a person, for instance during insertion or removal from a vaginal canal. It also preferably conveys to the person gripping the stent the orientation of the stent relative to a vaginal canal, such as in which direction to top of the stent faces. As used herein and with reference to FIG. 3, the “top” of the stent 10 means that the face of the stent 10 adjacent to the urethra 16, when the stent is properly and fully inserted into the vaginal canal, and the “bottom” of the stent means an opposite face of the stent adjacent to the rectum 19, when the stent is properly and fully inserted. References herein to the “side” of the stent 10 mean a side of the stent connecting the top and the bottom of the stent.

Various preferred features of transition depression portion 22 are as follows. Region 22 preferably extends over at least 80 percent of the top and sides of the stent, such as shown in FIGS. 1 and 2. However, as shown in FIG. 2, it is preferred that the bottom of stent 10 be substantially free of transition depression portion 22, where “substantially free” means that no more than about 20 percent of the bottom has a transition depression portion. This also aids in conveying to a person inserting the stent 10 into a vaginal canal the orientation of the stent, such as in which direction the top of the stent faces.

FIG. 3 illustrates a tilted view of the stent of FIG. 2, showing a top surface 50 and a bottom surface 52 of the stent and a shoulder region 26 of the stent.

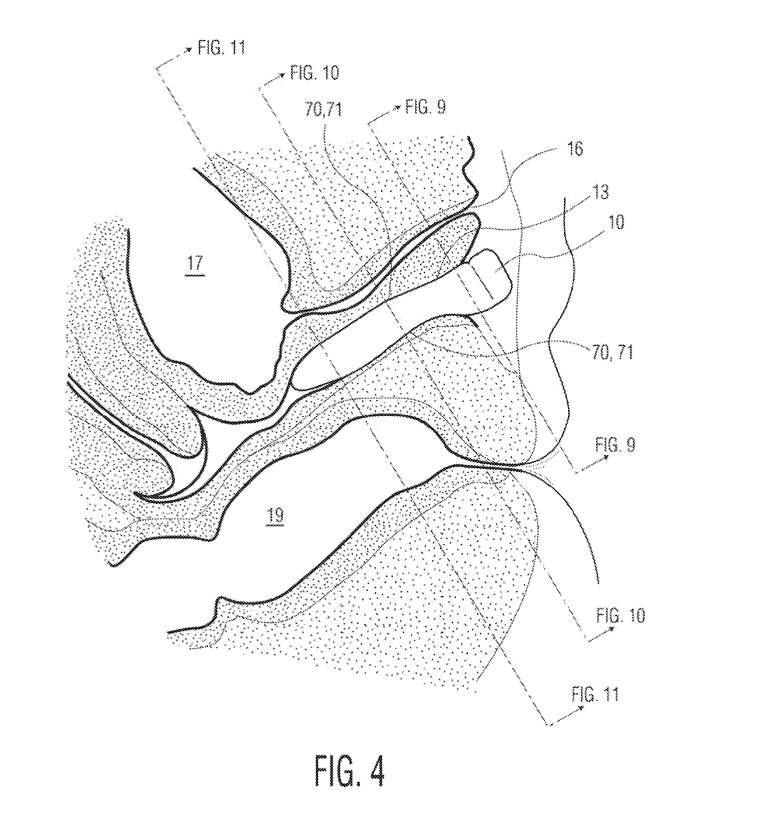

FIG. 5 illustrates other preferred features of knob portion 21. Preferably, the knob portion 21 is elongated in the top-to-bottom direction. Thus, knob portion 21 preferably has a top-to-bottom axis 30, intersecting an axis 29 of the body (best seen in FIG. 2), which exceeds a side-to-side axis 32 taken midway from top to bottom of knob portion 21, by 30 percent, and more preferably by 40 percent. Preferably, as shown in FIG. 4, the majority of each of the left- and right-shown sides 35 and 37 are approximately linear and are tapered towards each other in a direction from top to bottom, as shown by tapered phantom lines leading to an angle 77, which exceeds at least 6 degrees and may typically extend up to approximately 15 degrees. The foregoing elongated feature of the knob portion 21 and the tapering of the sides of the knob portion assist the user in identifying orientation of the vaginal stent 10 relative to the vaginal canal.

### Dimensions and Shaping of Body

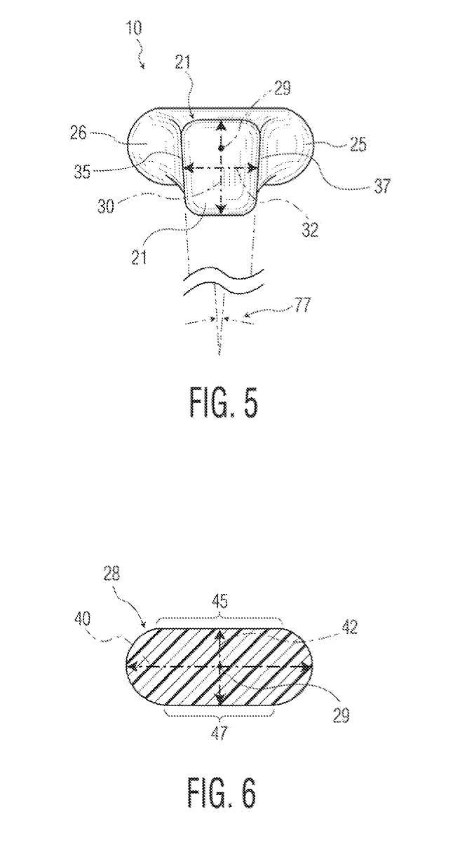

FIG. 6 shows a cross-section of stent body 10 of FIG. 1, taken mid-way along longitudinal axis 29 of the body. The cross-section shown in FIG. 5 defines a flatted shape having a major axis 40, which is approximately the largest left-to-right dimension in FIG. 5, and a minor axis 42, which is preferably taken midway along the major axis 40 and is orthogonal to the major axis. One advantage of the flattened shape as shown in FIG. 5 is that the shape is suitable to accommodate a corresponding flat transverse area of the vaginal canal. Variations on sizing, for example those for the flattened shape and cross-sectional areas of the stent, will be routine to a person of ordinary skill in the art as may be necessary, for example, in consideration of specific anatomy of a patient.

Referring to FIGS. 2 and 6, the length of stent 10, along longitudinal axis 29 of the body, may be about twice as large as the major axis, or width 40 (FIG. 5) of the stent. The minor axis 42 (FIG. 5) of the stent may typically be from ⅛ to ½ of the length of the stent (i.e., left-to-right dimension in FIG. 2). Selection of other dimension will be routine to persons of ordinary skill in the art based on the present specification.

In FIG. 6, the flatness of the illustrated cross-sectional shape of the body 21 is preferably defined by a top edge 45 of the body and a bottom edge 47 of the body, each centered about longitudinal axis 29 of the body and each having the length of the minor axis 42, being linear to within 0.5 mm or, alternatively, within 1 mm of a straight line.

Additionally, as shown in FIG. 2, the majority of the top surface 50 of the stent body 28 along the direction of longitudinal axis 29 may be approximately linear, and the bottom surface 52 of the stent body 28 in the same direction may be relatively more convexly curved as shown. Of course, the top and bottom surfaces 50 and 52 of the stent body 28 may be modified to fit the vaginal canal of a person.

Persons of ordinary skill in the will find it routine to modify the stent described herein to properly fit a person's vaginal canal. Such modifications may include altering the shape, size, curvature, dimensions, and materials of all parts of the invention, and may also include providing a user with variously sized and shaped stents, for'appropriate therapeutic relief.

### Stent Accommodations of Anatomy

Various configurations of the stent 10 accommodate anatomical features, as now explained. Thus, FIG. 2 shows the top surface of the neck portion 23 tapering from the body 28, in a direction from the distal (or right-shown end) to the proximal (or left-shown end) of the stent 10, towards the longitudinal axis 29 of the body. Such tapering avoids undue pressure on the urethra 16 (FIG. 4) when the stent if properly and fully inserted into a vaginal canal.

As also shown in FIG. 2, the bottom surface of the neck portion 21 preferably tapers, in a direction from the proximal (or left-shown) end of the stent 10 to the distal (or right-shown) end of the stent, towards said longitudinal axis to avoid undue pressure on the rectum (19, FIG. 4) when the stent is properly and fully inserted into a vaginal canal.

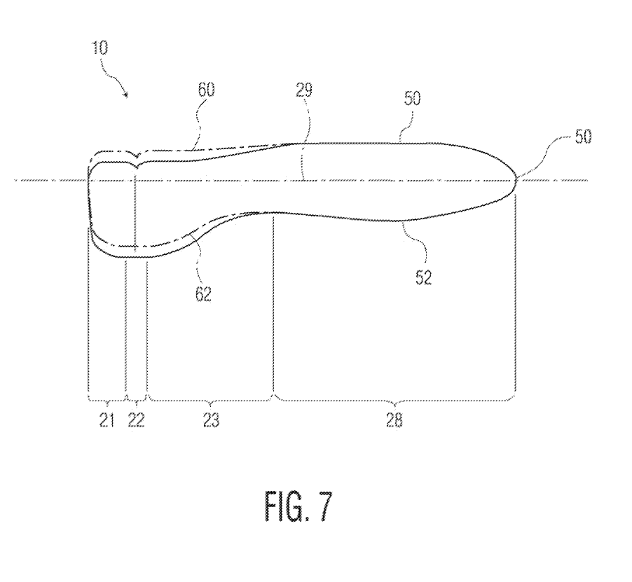

FIG. 7 shows a stent 10 with modifications to the curvature described in the foregoing three paragraphs, in which the numeral 60 shows an alternative upper neck contour, and numeral 62 shows an alternative lower neck contour. These alternative contours are among many alternative contours that may be used, as will be routine to persons of ordinary skill in the art depending on such factors as person anatomy, comfort, and ease of insertion.

Stents that lack the curvature described in the foregoing two paragraphs may result in undue pressure to the urethra and rectum as described. Such undue pressure causes discomfort to a person, and will likely reduce a person's willingness to continue treatment with the stent. Stents lacking the curvature described in the foregoing two paragraphs may also result in undue pressure to the urethra or rectum or both during the process of insertion and removal of the stent from a vaginal canal.

The curvature of neck portion 21 may be further modified as will be routine to a person of ordinary skill in the art as necessary, for example, in consideration of specific anatomy of a patient.

### Insertion and Removal of Vaginal Stent

Aspects of the present invention also extend to methods of insertion and removal of the vaginal stents described above. To better understand these methods, FIGS. 7-10 help explain preferred methods of insertion of a stent into a vaginal canal.

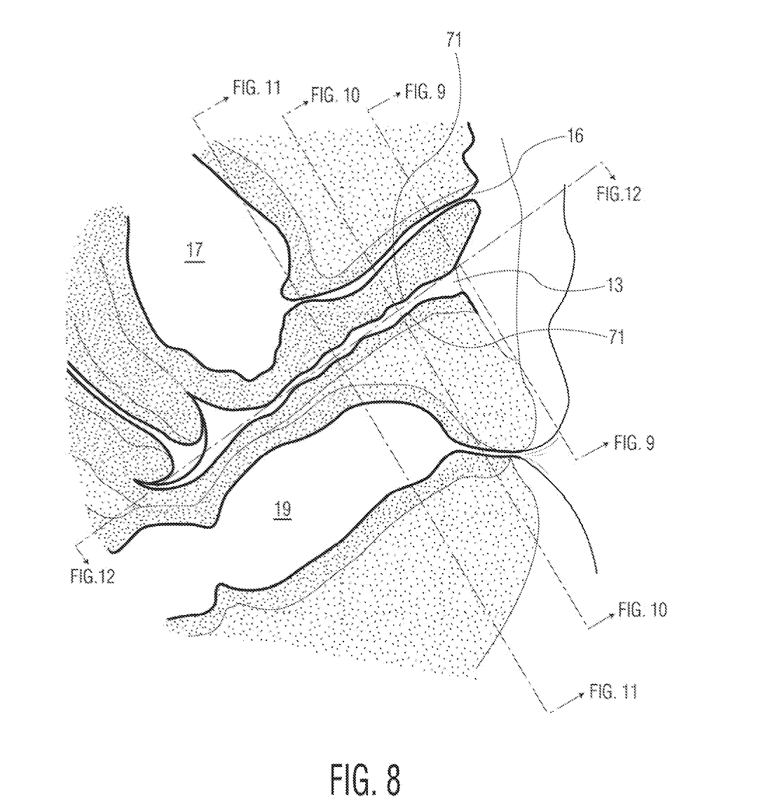

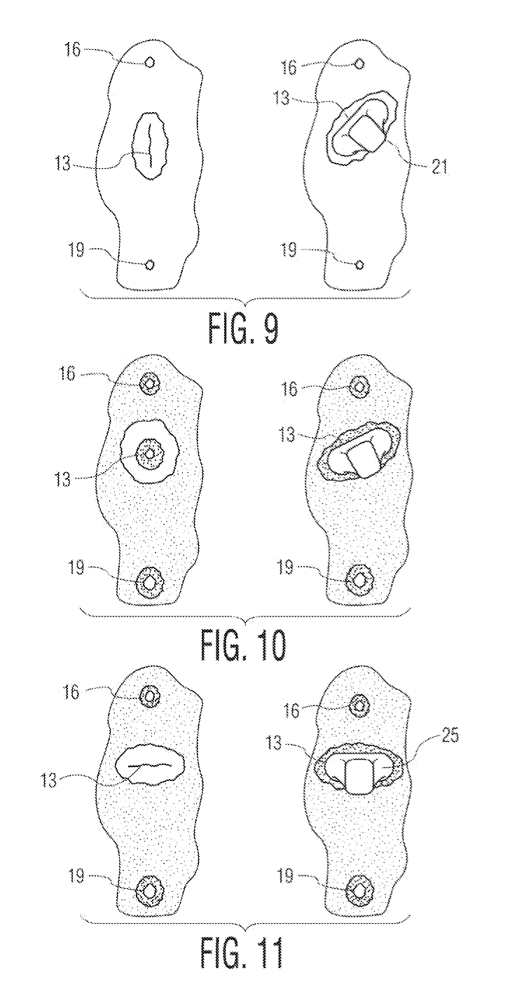

In particular, FIG. 8 shows a vaginal canal 13 and adjacent anatomic organs, such as a urethra 16 and associated urinary bladder 17 above the vaginal canal 13, and a rectum 19 below the vaginal canal. In FIG. 8, the arrows marked FIG. 9, FIG. 10 and FIG. 11 show progressively different depths into the vaginal canal, which are respectively illustrated in FIGS. 8-10.

Thus, FIG. 9 shows on the left a vaginal canal 13, from outside the vaginal canal and the relative positions of the urethra 16 and the rectum 19. The empty vaginal canal is shown closed, and is typically closed for the first in the first 2 cm of depth in a mature female person. The orifice, or opening, of the vaginal canal is shown with a generally linear orifice, which is vertically oriented in FIG. 9. Such generally linear orifice is referred to herein as an approximately linear opening.

The right side of FIG. 9 shows the knob portion 21 of the stent inserted into vaginal canal 13 with its major axis (40, FIG. 6) at a typical angle of 45 degrees from the approximately linear opening of the vaginal canal 13 as shown on the left of FIG. 9. This is to prevent undue pressure from the stent pressing on the urethra 16 during insertion or removal of the stent. Instead of the foregoing angle of 45 degrees, other angles, and preferably angles between 30 and 60 degrees, and more preferably angles between 40 and 50 degrees, can avoid undue pressure on the urethra. Thus, a first step of a preferred method of inserting a vaginal stent is to pass the distal end of a stent into the vaginal orifice at an angle according to the foregoing description.

The left side shown of FIG. 10, showing cross sections of FIG. 8 at a greater depth into the vaginal canal 13 than the above-discussed FIG. 9, shows the vaginal canal 13 neither as linear in the illustrated vertical direction nor as linear in the illustrated horizontal direction, but more like a general circular shape that can assume different orientations when the vaginal stent is inserted into the vaginal canal, for instance.

On the right side of FIG. 10, the stent is shown in a typically further rotated position, in which the stent is at an angle of 60 degrees, for instance, from the illustrated vertical direction. The stent's rotation from the position shown in FIG. 9 to the position shown in FIG. 10 may be due to manipulation by a user, or at least partly or fully by self-rotation due to anatomical features such as the presence of the levator ani muscles of the pelvic floor, located in the vicinity of the stippled region 70, more inwardly in the vaginal canal 13 than the cross section of FIG. 10.

On the left side of the progressively deeper cross section of FIG. 8 shown in FIG. 11, the vaginal canal 13 is oriented mostly linearly in the illustrated horizontal direction. On the right side of FIG. 11, the stent is shown with the major axis (50, FIG. 4) aligned with the illustrated horizontal direction. In this position, shoulders 25 and 26 of the stent are self-retained in the vaginal canal 13 due to support provided by pelvic floor muscles such as the levator ani muscles in region 70 (FIG. 8) and associated fascia (not shown).

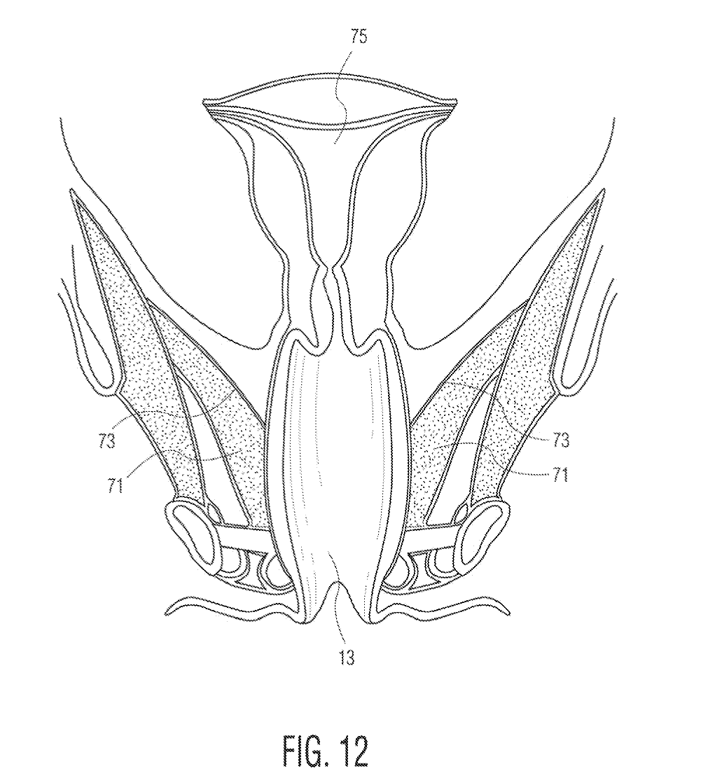

FIG. 12 further shows the levator ani muscles 71 and associated superior and inferior pelvic diaphragmatic fascia 73, which supports the vaginal canal 13, shown extending from a vaginal orifice at the bottom of the figure to a uterus 75. As mentioned, it is the levator ani muscles 71 that retain the shoulders 25 and 26 (FIG. 11) of the stent in position within the vaginal canal 13.

Preferably, the stent is sized so that, when the shoulders 25 and 26 (FIG. 10) of the stent are retained by the levator ani muscles 71 (FIG. 7), approximately only the neck portion 21 and the transition depression portion (22, FIGS. 1 and 2) protrude from the orifice of the vaginal canal 13. For an adult female, this will mean that a typical length of about 8-10 cm of the stent protrudes into the vaginal canal 13. This is a helpful guide for determining how deeply the stent should be inserted into the vaginal canal.

The shoulders of the stent help allow the stent to be self-retaining for several hours, in one example.

Without bound by theory, the vagina is attached laterally to the arcus tendineus fascia pelvis and to the levator ani muscle, which comprises three muscles, iliococcygygeus, puborectalis and pubococcygeus muscles.

Rotation of the stent from the position shown on the right of FIG. 10 to the right of FIG. 11 may occur by manipulation by a person or may occur at least partially or fully by self-rotation owing to the forces places on the stent by, for instance, the levator ani muscles 71 (FIG. 12) located in region 70 of FIG. 8.

Removal of a stent from a vaginal canal occurs in one example (not illustrated), by placing an index finger and thumb around the knob portion with part of the finger and thumb preferably entering a transition depression portion, and then rotating the knob portion to dislodge the shoulders of the stent from the support given by the levator ani muscles and associated fascia, and pulling the stent out of the vaginal canal at preferably the same angles discussed above for insertion of the stent.

### Benefits of Use of Vaginal Stent

The above-described stent is particularly useful for post-menopausal women who experience decreases in width and depth of the vaginal canal, and more particularly post-menopausal women who never had children.

The stent is also useful in dilating the vaginal canals of woman who undergo radiation treatment for cancer and who therefore need to use vaginal dilators regularly.

The stent may also be particularly helpful for women with congenital disorders. For example, women of all ages, who have congenital abnormalities in the formation of the vaginal canals, such as I-Rokitansky syndrome (MRKH syndrome) may use the stent in order to develop a vagina. If later surgery is required, the patient may use the vaginal stent post-operatively to maintain a state of the vaginal canal being open and to avoid secondary stricture.

The stent may also be used as vaginal dilators for women who have abnormally small vaginal canals, so as to avoid the need for surgery.

The stent is useful in reducing the spasms of women with vaginismus, and who experience severe pain and spasm of the vagina due to intense anxiety.

The stent can be used in gradually increasing sizes to help dilate the vagina and to avoid dyspareunia, or painful sexual intercourse.

The following is a list of reference numerals and associated parts as used in this specification and drawings:

| Reference Numeral | Part |
|---|---|
| 10 | Stent |
| 13 | Vaginal canal |
| 16 | Urethra |
| 17 | Urinary bladder |
| 19 | Rectum |
| 21 | Knob portion |
| 22 | Transition depression portion |
| 23 | Neck portion |
| 25 | Shoulder region |
| 26 | Shoulder region |
| 28 | Body |
| 30 | Top-to-bottom axis |
| 32 | Side-to-side axis |
| 35 | Side |
| 37 | Side |
| 38 | Stent end |
| 40 | Major axis |
| 42 | Minor axis |
| 45 | Top edge |
| 47 | Bottom edge |
| 50 | Top surface |
| 52 | Bottom surface |
| 60 | Alternative upper neck contour |
| 62 | Alternative lower neck contour |
| 70 | Region of levator ani muscles |
| 71 | Levator ani muscles |
| 73 | Fascia |
| 75 | Uterus |
| 77 | Angle |

While the invention has been described with respect to specific embodiments by way of illustration, many modifications and changes will occur to those skilled in the art. It is, therefore, to be understood that the appended claims are intended to cover all such modifications and changes as fall within the true scope and spirit of the invention.

What is claimed is:

1. A vaginal stent having a top and a bottom and comprising, from a proximal end of the stent to a distal end of the stent, a knob portion, a neck portion including two shoulder regions, and a body with a top surface and a bottom surface; a periphery of a cross section of the body taken midway along the body along a longitudinal axis of the body, in a direction from the proximal end to the distal end of the stent, defining a flattened shape having a major axis and a minor axis.

2. The vaginal stent of claim 1, wherein the top surface of the neck portion tapers from the body, in a direction from the distal end to the proximal end of the stent, towards said longitudinal axis, so as to avoid undue pressure on the urethra when the stent is properly and fully inserted into a vaginal canal.

3. The vaginal stent of claim 1, wherein the bottom surface of the neck portion tapers, in a direction from the proximal end to the distal end of the stent, towards said longitudinal axis to avoid undue pressure on the rectum when the stent is properly and fully inserted into a vaginal canal.

4. The vaginal stent of claim 1, wherein the neck portion tapers away from the shoulders towards the knob.

5. The vaginal stent of claim 1, wherein the ratio of the major axis of the flattened shape to the minor axis of the flattened shape is at least 2:1.

6. The vaginal stent of claim 1, wherein the stent further comprises a transition depression portion coupling the knob portion to the neck portion.

7. The vaginal stent of claim 6, wherein the transition depression portion extends over at least 80 percent of the top and sides of the stent.

8. The vaginal stent of claim 7, wherein the bottom of the knob is substantially free of a transmission depression portion.

9. The vaginal stent of claim 1, wherein the knob is elongated from top to bottom of the stent.

10. The vaginal stent of claim 9, wherein a cross section of the knob along said longitudinal axis has a top-to-bottom axis and an orthogonal, side-to-side axis measured midway between the top and the bottom of the knob, the top-to-bottom axis exceeding the side-to-side axis by at least 30 percent.

11. The vaginal stent of claim 9, wherein a cross section of the knob portion along said longitudinal axis has a top-to-bottom axis and an orthogonal, side-to-side axis measured midway between the top and the bottom of the knob, the top-to-bottom axis exceeding the side-to-side axis by at least 40 percent.

12. The vaginal stent of claim 9, wherein, in a cross section of the knob portion along said longitudinal axis, respective majorities of a pair of sides of the knob portions are approximately linear and taper towards each other in a top-to-bottom direction.

13. The vaginal stent of claim 12, wherein the majority of the left and right sides taper towards each other at an angle exceeding 6 degrees.

14. The vaginal stent of claim 1, wherein the flatness of said shape is defined by a top edge of the body and a bottom edge of the body, each centered about said longitudinal axis and each having the length of the minor axis, being linear to within 0.5 mm of a straight line.

15. The vaginal stent of claim 1, wherein the material comprising the stent is of a plastic, acrylic, or silastic material, and combinations thereof.

16. The vaginal stent of claim 1, wherein the distal end of the stent is configured to be inserted into the vaginal canal such that the shoulder region allows the stent to be self-retaining in the vaginal canal due to the support given by levator ani muscles and fascia.

17. A method of using the vaginal stent of claim 1, comprising:
a) first inserting the distal end of the stent into the a vaginal orifice of a human female with said major axis of the stent body being sufficiently angled from an approximately linear orifice of an empty vagina such that the knob avoids pressing a female urethra;
b) further moving the stent backwardly into the vaginal canal until the neck portion becomes positioned proximate the vaginal orifice and the stent extends for a length into the vaginal canal such that the stent becomes locked in place by having the shoulders of the neck portion being self-retained and supported by levator ani muscles and fascia of the vaginal canal; and
c) further positioning the stent such that the stent is substantially horizontally aligned with the transverse area of the vaginal canal by rotating of the knob.

18. The method of claim 17, wherein, during said first inserting the distal end of the stent into a vaginal orifice, said major axis is sufficiently angled from the approximately linear orifice to avoid undue pressure on a urethra.

19. The method of claim 17, wherein, during said first inserting the distal end of the stent into a vaginal orifice, said major axis is angled from the approximately linear orifice at an angle between 30 and 60 degrees.

20. The method of claim 19, wherein, during said first inserting the distal end of the stent into a vaginal orifice, said major axis is angled from the approximately linear orifice at an angle between 40 and 50 degrees.

21. The method of claim 17, wherein, during the step of further positioning the stent, the rotating of the knob occurs on its own, due to forces acting on the vaginal canal and support from the levator ani muscles and the fascia, and such rotating is sufficient for final positioning of the stent.

22. The method of claim 21, wherein, during the step of further positioning the stent, the rotating of the knob is not sufficient for the final positioning, and the rotating of the knob is further facilitated by a person, after the step of rotating the knob on its own.

23. The method of claim 17, wherein, during the step of further positioning the stent, the rotating of the knob is performed by a person.

24. The method of claim 17, wherein, during the step of further positioning the stent, the proximal end of the knob portion protrudes outside the vaginal canal from one to two centimeters.

25. The method of claim 17, further comprising the step of removing the stent by having a person place an index finger and thumb around the knob, and rotating the knob portion a sufficient amount to dislodge the stent from support given by the levator ani muscles and the fascia and allow for pulling of the stent out of the vaginal orifice.
